(19) United States
(12) Patent Application Publication
(10) Pub. No.: US 2009/0181358 A1
(43) Pub. Date: Jul. 16, 2009

(54) Marker for Chromosomal Abnormalities

(76) Inventor: Susan J. Gross, Bronx, NY (US)

Correspondence Address: Craig J Arnold;Amster Rothstein & Ebentein, 90 Parl Avenue, New York, NY, 10016, US

(21) Appl. No.: 12/224,683

(22) PCT Filed: Mar. 27, 2007
(86) PCT No.: PCT/US07/07742

§ 371 Date: Dec. 8, 2008

Related U.S. Application Data
(60) Provisional application No. 60/788,388, filed on Mar. 30, 2006.

(51) Int. Cl. C12Q 1/00

(52) U.S. Cl. 435/4

(57) ABSTRACT

Provided are methods of determining whether a pregnant woman is at elevated risk for carrying a fetus with a chromosomal abnormality. Also provided are kits for determining whether a pregnant woman is at elevated risk for carrying an aneuploid fetus.

### CROSS-REFERENCE TO RELATED APPLICATION

This application claims the benefit of U.S. Provisional Application No. 60/788,388, filed Mar. 30, 2006.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grants R03 HD40342-01 awarded by The National Institute of Child Health and Human Development.

### BACKGROUND OF THE INVENTION

(1) Field of the Invention

The present invention generally relates to markers for predicting fetal abnormalities. More specifically, the invention provides a marker useful for determining whether a pregnant woman is at elevated risk for carrying an aneuploid fetus.

(2) Description of the Related Art

Trisomy 21, commonly referred to as Down syndrome (DS), is the most frequent chromosome disorder seen in newborns, and is the leading genetic cause of moderate mental retardation (Thompson et al., 1991). With the advent of amniocentesis in the early 1970s, testing for this disorder during pregnancy became feasible (NICHD National Registry, 1976). Nevertheless, amniocentesis was never offered to all women on a population wide basis, as it is associated with a recognized fetal loss rate of 1/200. Thus, normal pregnancies can be lost due to the procedure itself. Consequently, amniocentesis is only considered standard of care for women who are 35 years of age and older, as this population is considered a high-risk group for chromosomal disorders (Hook, 1981). However, as a result of this policy, the majority of DS children in the US are born to younger mothers, as this is the population group most likely to conceive and have offspring. Consequently, researchers have continued to seek a non-invasive, safe method of detection or at a minimum, improved screening, in which case a high-risk cohort among a low-risk population could be identified and that group could then be offered invasive testing.

In 1984, low concentrations of α-fetoprotein (AFP) in maternal serum was correlated with fetal DS (Merkatz et al., 1984), presaging the first serum screening test for DS. Additionally, for several years, maternal serum screening using elevated levels of alpha-fetoprotein (AFP), a protein produced by the fetal liver, has been used as a screening test for fetal neural tube defects. The ability to use serum AFP concentration combined with maternal age as a screening test for DS was confirmed and brought into general practice over the subsequent years (New England Regional Genetics Group Prenatal Collaborative Study, 1989). Further serum markers have subsequently been added, including human chorionic gonadotropin (hCG) which is elevated (Bogart et al., 1987), unconjugated estriol (uE3), which is decreased (Canick et al., 1988) and inhibin A which is also elevated (Cuckle et al., 1996). See also Nicolaides et al., 2005; Wald et al., 2000; U.S. Pat. Nos. 4,874,693; 5,316,953; and 6,025,149; and International Patent Publication Nos. WO 90/08325; WO 94/03804; and WO 00/40428. In combination with maternal age, these second trimester markers can detect approximately 70% to 80% of cases of fetal DS and multiple marker serum screening in pregnant women under 35 is considered standard of care in this country (The American College of Medical Genetics Policy Statement and provides an excellent summary of the current state of serum screening in this country, as well as recommendations). Despite considerable improvements over this past decade, the serum screening method still entails a significant false positive rate. Major National Institute of Child Health and Human Development-funded studies have now been completed whereby testing can be performed in the first trimester. This latest approach has been to combine fetal ultrasound with additional markers between gestational weeks 10 and 13 (free beta-hCG and PAPP-A) and a new marker, ADAM 12, has recently been reported (Laigaard et al., 2003). Due to the number of pregnant women affected, and the serious ramifications with respect to reproductive health care, this issue of improved screening is a priority at the national level.

Ultimately, serum markers are only a screening tool and not a diagnostic test. Thus, when patients are screened positive, they are offered amniocentesis to confirm the suspicion of fetal DS. The vast majority of patients who undergo amniocenteses in such circumstances will be carrying normal fetuses. There is nothing specific about these markers with respect to fetal chromosomal abnormalities and were primarily discovered in a context distinct from aneuploidy and many were initially used in other clinical settings (e.g., AFP and neural tube defects, as mentioned above).

The addition of new markers for serum screening would allow for earlier detection and could potentially be combined with second trimester results to enhance detection and specificity, ultimately reducing the number of false positives (unnecessary invasive tests) and false negatives (missed diagnoses).

One approach to identifying additional Down markers is to use microarray technology to identify genes that are overexpressed, e.g., in placenta (Gross et al., 2002). That approach was based on the hypothesis that one or more genes may be turned on during development (thus explaining the characteristic dysmorphology and mental retardation). This suggests that the DS phenotype is the result not of specific chromosome 21 gene overexpression, but rather a non-specific disorder of chromosome balance, which results in abnormal homeostasis (Shapiro, 1999). Furthermore, studies indicate that gene expression will vary between different tissues (Holtzman et al., 1992), particularly placenta, which would be expected to tolerate greater alterations in genetic expression when compared to fetal tissue. Placenta also proved an attractive tissue type to study due to its close proximity to the maternal circulation, which allows protein produced by placental tissue to cross over into the maternal circulation.

Gross et al. (2002) identified several genes that are overexpressed during Down syndrome. Protein expression of one of these, keratin 8, was later confirmed to be elevated in Down placentas (Klugman et al., 2004). It would be desirable to determine whether any of the genes identified in Gross et al. (2002), could be used as a bodily fluid (e.g., serum, urine) marker for Down syndrome, either alone or in combination with other markers to reduce false positive and false negative rates of Down syndrome screens. The present invention addresses that need.

### SUMMARY OF THE INVENTION

Accordingly, the inventor has discovered that pregnant women with elevated tissue polypeptide antigen (TPA) levels in bodily fluids have an elevated risk of carrying an aneuploid fetus.

Thus, the invention is directed to methods of determining whether a pregnant woman is at elevated risk for carrying a fetus with a chromosomal abnormality. The methods comprise

(a) obtaining a biological fluid sample from the woman;

(b) determining the concentration of tissue polypeptide antigen (TPA) in the biological fluid sample; and

(c) using the concentration determined in step (b) for determining whether the pregnant woman is at elevated risk for carrying a fetus with a chromosomal abnormality.

The invention is also directed to kits for determining whether a pregnant woman is at elevated risk for carrying a fetus with a chromosomal abnormality. The kits comprise a first detectable agent specific for tissue polypeptide antigen (TPA) or a component thereof and

a second detectable agent specific for α-fetoprotein (AFP), human chorionic gonadotropin (hCG), unconjugated estriol (uE3), pregnancy-associated plasma protein A (PAPP-A), proform of eosinophilic major basic protein (proMBP), total estrogen, total estriol, or inhibin A.

### DETAILED DESCRIPTION OF THE INVENTION

The inventor has discovered that tissue polypeptide antigen (TPA) concentration is generally elevated in bodily fluids of women carrying a fetus having Down syndrome and reduced in bodily fluids of women carrying a fetus having other chromosomal abnormalities. See Example. While TPA has been measured in bodily fluids of pregnant women (Itahashi et al., 1988; Bremme et al., 1985; Bancher-Todesca et al., 2001; Inaba et al., 1993; Schrocksnadel et al., 1993; Lelle et al., 1989; Jamfelt-Samsioe et al., 1986; Bergant et al., 1996), it has not previously been established that TPA concentration in bodily fluids correlates with any fetal abnormality. Based on this discovery, TPA measurement in a bodily fluid of a pregnant woman can be used to determine whether the pregnant women is at elevated risk for carrying a fetus having a chromosome abnormality.

Thus, the invention is directed to methods of determining whether a pregnant woman is at elevated risk for carrying a fetus with a chromosomal abnormality. The methods comprise

(a) obtaining a biological fluid sample from the woman;

(b) determining the concentration of tissue polypeptide antigen (TPA) in the biological fluid sample; and

(c) using the concentration determined in step (b) for determining whether the pregnant woman is at elevated risk for carrying a fetus with a chromosomal abnormality.

As used herein, a chromosomal abnormality is a condition where there is a structural or numerical alteration from a normal chromosomal complement, where the alteration can be visualized using cytogenetic techniques such as karyotyping. Chromosomal abnormalities include aneuploidy (including but not limited to 47, XYY; trisomy 21 [Down syndrome]; trisomy 18 [Edwards syndrome]; trisomy 13 [Patau syndrome]; 47, XXY [Klinefelter's syndrome]; monosomy 18; 45, X [Turner syndrome]; and 47, XXX), triploidy, tetraploidy, inversions (including paracentric and pericentric inversions), translocations (including reciprocal and Robertsonian translocations), deletions (including terminal [telomeric] and interstitial deletions, cri du chat syndrome [5p-syndrome], and Wolf-Hirschhom syndrome [4p-deletion]), insertions, duplications, ring chromosomes, isochromosomes, or mosaics having cells comprising any of the above abnormalities.

The determination step (c) of these methods can utilize any means of determining whether the concentration of TPA in the sample is elevated. Preferably, that step involves comparing the concentration determined in step (b) with control concentration(s) from pregnant women that are not carrying fetuses with a chromosomal abnormality. Here, if the TPA concentration in the biological fluid sample is greater than the average control concentration, then the pregnant woman has an elevated risk of carrying a fetus with Down syndrome. Also, if the TPA concentration in the biological fluid sample is less than the average control concentration, then the pregnant women has an elevated risk of carrying a fetus with a chromosomal abnormality other than Down syndrome. Additionally, if the TPA concentration in the biological fluid sample is not greater or less than the average control concentration, then the pregnant women does not have an elevated risk of carrying a fetus with a chromosomal abnormality.

A skilled artisan can design and execute the collection of the control fluid and measurement of the TPA concentration in that control fluid without undue experimentation. Preferably, the control fluid is matched as much as possible with the sample fluid, e.g., in fluid type, age of the pregnant women, gestational phase of the pregnant women and similar general health. The control TPA concentration is also preferably an average of measurements of control fluids from multiple women such that a measurement of variation in TPA concentration among control fluids can be ascertained, using known statistical tools.

As used herein, “greater than” or “less than” the average TPA control concentration means a difference in concentration that is different enough from the control concentration(s) to achieve a desired detection level and/or false positive rate. These values can be determined by the skilled artisan without undue experimentation using well-known statistical tools. In some cases, these statistical goals can be achieved though multiple sampling, e.g., at weekly intervals, or retesting after achieving a significant difference from the control value.

Preferably, these methods are used to evaluate whether the woman is carrying an aneuploid fetus, most preferably Down syndrome.

It is expected that the methods of the present invention can utilize any biological fluid including but not limited to saliva, bile, lymph, mucus, or, preferably, urine or peripheral blood serum, or matched samples of each. Peripheral blood serum is most preferred, since other tests for aneuploidies most often utilize maternal serum, so the same serum sample can conveniently be used in the method of the present invention and in other screening tests for chromosomal abnormalities.

If the invention method shows an elevated risk for an aneuploid fetus, the method preferably further comprises subjecting the woman to further testing for carrying an aneuploid fetus. The further testing can be any test that can more definitively determine whether the pregnant woman is carrying an aneuploid fetus. Such tests include high-resolution sonogram screening or preferably amniocentesis or chorionic villus sampling.

These methods can also include tests for other known markers of chromosome abnormalities, such that the combination can achieve a higher detection level and/or a lower false positive rate than either test alone. Nonlimiting examples of such tests include determining the concentration a-fetoprotein (AFP), human chorionic gonadotropin (hCG), unconjugated estriol (uE3), pregnancy-associated plasma protein A (PAPP-A), proform of eosinophilic major basic protein (proMBP), total estrogen, total estriol, and/or inhibin A in the biological fluid sample. Preferably, the concentrations of AFP, hCG, uE3 and inhibin A are determined along with TPA.

The methods of the present invention can be used with a woman is in the first trimester or in the second trimester of pregnancy (see Example).

The concentration of TPA can be determined by measuring total TPA with an assay designed to detect TPA (e.g., as made by Sangtec Medical Co, Bromma, Sweden) or by determining the concentration of a component of TPA in the sample TPA is a molecular complex containing cytokeratins 8, 18 and 19 that is used as a cancer marker (Weber et al., 1984; Sundstrom and Stigbrand, 1994; Barak et al. 2004). The preferred TPA component is keratin 8.

The invention is also directed to kits for determining whether a pregnant woman is at elevated risk for carrying an aneuploid fetus. The kits comprise

a first detectable agent specific for tissue polypeptide antigen (TPA) or a component thereof and

a second detectable agent specific for α-fetoprotein (AFP), human chorionic gonadotropin (hCG), unconjugated estriol (uE3), pregnancy-associated plasma protein A (PAPP-A), proform of eosinophilic major basic protein (proMBP), total estrogen, total estriol, or inhibin A. Preferably, the kit comprises detectable agents specific for AFP, hCG, uEA and inhibin A.

The kits of the present invention are not limited to any particular detectable agent specific for TPA, AFP, hCG, uE3, PAPP-A, proMBP, total estrogen, total estriol and/or inhibin A. Nonlimiting examples of such useful agents include aptamers and naturally occurring proteins that bind or otherwise interact with TPA, AFP, hCG, uE3, PAPP-A, proMBP, total estrogen, total estriol or inhibin A. Preferably, the agent comprises an antibody binding site that is specific for TPA, AFP, hCG, uE3, PAPP-A, proMBP, total estrogen, total estriol or inhibin A since proteins containing specific antibody binding sites can be routinely generated by a number of methods known in the art. It is thus preferred that all of the detectable agents comprise an antibody binding site, most preferably antibodies.

The kits are also not limited to any particular method of detecting the detectable agents. Such methods can include, for example, induction of a visible change to living cells in the assay (e.g., induction or suppression of apoptosis), utilization of a radioactive, enzymatic or fluorescent label on the detectable agent (e.g., antibody), or on a second agent that specifically binds to the first agent (e.g., second antibody), etc. Myriad such methods are known in the art.

The first detectable agent is preferably an antibody preparation that is specific for TPA. The antibody preparation can be any preparation comprising an antibody binding site specific fur TPA, e.g., monoclonal antibody, polyclonal antibody, recombinant antibody or other proteins comprising an antibody binding site including single chain antibodies. The first detectable agent can also be specific for a component of TPA, preferably keratin 8.

Preferred embodiments of the invention are described in the following example. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the example.

### EXAMPLE

### Correlation of Tissue Polypeptide Antigen Concentration with Down Syndrome and Other Aneuploid Disorders

Experimental design:

In Gross et al. (2002), several genes were found to be over-expressed in Down syndrome (DS) placentas, which was confirmed by Northern blot analysis and quantitative real time RT-PCR (QPCR) was keratin 8 (cytokeratin 8; KRT8). Keratins are the largest subgroup of the intermediate filaments. KRT8, along with keratin 18, is an intermediate filament that is associated with differentiation to the trophoectodermal layer of the blastocyst. In addition to its role as a structural protein in differentiation, KRT8 is involved in signal transduction and potentially apoptosis. To determine whether KRT8 is a useful marker for Down syndrome and other diseases characterized by aneuploidies, an assay kit for tissue polypeptide antigen (TPA) produced by Sangtec, Sweden was used to evaluate serum samples from 311 women who underwent normal pregnancies, 64 samples from pregnant women carrying a DS and 30 other serum samples from pregnancies with chromosomal abnormalities other than DS were assessed.

Statistics. Mathematical algorithms to determine DS risk are calculated by determining the medians for the normal population and the Down syndrome population. The greater the difference in the medians, and the smaller the standard deviations for the curves, the more useful the marker. Multiples of the median are used in order to standardize the findings.

### Results.

The following are the overall MoMs (multiples of the median):
Down syndrome: 1.37
   Other chromosomal abnormalities (trisomy 18; trisomy 13; Klinefelter's syndrome; monosomy 18; karyotype 45, X; karyotype 47, XXX; other trisomies): 0.69
Normal: 1.00
   The DS versus normal difference is highly significant (P<0.0001) as are the other chromosomal abnormalities (P=0.001).
   Breaking it down by trimester gives median MoMs:
1T (first trimester) DS (n=13) 1.51
1T normal (n=76) 1.03
2T (second trimester) DS (n=51) 1.28
2T normal (n=235) 0.99
   The 1T difference is significant as a 1-tail comparison (P=0.03) despite the small numbers. The 2T difference is still highly significant at 2-tail (P<0.0001).
   In both DS and normals there are correlations between TPA and free beta or hCG (depending on trimester) and between TPA and PAPP-A.
   Regressing the weekly median MoM there is a trend ( P=0.20) The regressed medians are:
11 weeks 1.78 MoM
12 weeks 1.67 MoM
13 weeks 1.57 MoM
14 weeks 1.47 MoM
15 weeks 1.38 MoM
16 weeks 1.30 MoM
17 weeks 1.22 MoM
18 weeks 1.14 MoM

### REFERENCES

ACOG Educational Bulletin, No. 228, September 1996—Maternal Serum Screening Shapiro BL. The Down syndrome critical region. J Neural Transm Suppl. 1999; 57:41-60.

Bancher-Todesca D et al. Hypertens Pregnancy 2001; 20:89-98.

Barak V et al. Clin. Biochem. 2003; 37:529-40.

Bergant A et al. Gynecol Obstet Invest 1996; 41:237-9.

Bogart M H, Pandian M R, Jones O W. Abnormal maternal serum chorionic gonadotropin levels in pregnancies with fetal chromosome abnormalities. Prenat Diagn. 1987 November; 7(9):623-30.

Bremme K et al. Gynecol obstet Invest 1985; 19:118-23.

Canick J A, Knight G J, Palomaki G E, Haddow J E, Cuckle H S, Wald N J. Low second trimester maternal serum unconjugated oestriol in pregnancies with Down's syndrome. Br J Obstet Gynaecol. 1988 April; 95(4):330-3.

Cuckle H S, Holding S, Jones R, Groome N P, Wallace E M. Combining inhibin A with existing second-trimester markers in maternal serum screening for Down's syndrome. Prenat Diagn. 1996 December; 16(12):1095-100.

Gross S J, Ferreira J C, Morrow B, Dar P, Funke B, Khabele D, Merkatz I. Gene expression profile of trisomy 21 placentas: a potential approach for designing noninvasive techniques of prenatal diagnosis. Am J Obstet Gynecol 2002 August; 187(2):457-62.

Holtzman D M, Bayney R M, Li Y W, Khosrovi H, Berger C N, Epstein C J, Mobley W C. Dysregulation of gene expression in mouse trisomy 16, an animal model of Down syndrome. EMBO J. 1992 February; 11(2):619-27.

Hook E B. Rates of chromosome abnormalities at different maternal ages. Obstet Gynecol 1981 September; 58(3):282-5.

Inaba N et al. Asia Oceana J Obstet Gynaecol 1993; 19:459-66.

Itahashi K et al. Arch Gynecol Obstst 1988; 243:191-7.

Jarnfelt-Samsioe A et al. Acta Obstst Gynecol Scand 1986; 65:745-51.

Klugman S et al. Expression of Keratin 8 and TRAIL in Down Syndrome placentae. American Society of Human Genetics 54^{th} Annual Meeting Abstract No. 180, 2004.

Laigaard J, Sorensen T, Frohlich C, Pedersen B N, Christiansen M, Schiott K, Uldbjerg N, Albrechtsen R, Clausen H V, Ottesen B, Wewer U M. ADAM12: a novel first-trimester maternal serum marker for Down syndrome. Prenat Diagn. 2003 Dec. 30; 23(13):1086-91.

Lelle R J et al. Gynecol Obstst Invest 1989; 27:137-42.

Merkatz I R, Nitowsky H M, Macri J N, Johnson W E. An association between low maternal serum alpha-fetoprotein and fetal chromosomal abnormalities. Am J Obstet Gynecol. 1984 Apr. 1; 148(7):886-94.

New England Regional Genetics Group. Combining maternal serum alpha-fetoprotein measurements and age to screen for Down syndrome in pregnant women under age 35. New England Regional Genetics Group Prenatal Collaborative Study of Down Syndrome Screening. Am J Obstet Gynecol. 1989 March; 160(3):575-81.

NICHD National Registry for Amniocentesis Study Group, Midtrimester amniocentesis for prenatal diagnosis: safety and accuracy. JAMA 1976; 236:1471-6.

Nicolaides K H et al. Ultrasound in Obstet Gynecol 2005; 25:221-6.

Schrocksnadel H et al. Gynecol Obstet Invest 1993; 35:204-8.

Sundstrom B E, Stigbrand T I. Int J Biol Markers 1994; 9:102-8.

Thompson M W, McInnes R R, Willard H F. (1991). Genetics in medicine. 5th ed. W B Saunders, Philadelphia, p.219.

Wald N J et al. Obstetrical Gynecological Survey 2000; 55:10-1.

Weber K et al. EMBO J. 1984; 3:2707-14.

U.S. Pat. No. 4,874,693.

U.S. Pat. No. 5,316,953.

U.S. Pat. No. 6,025,149.

International Patent Publication No. WO 90/08325.

International Patent Publication No. WO 94/03804.

International Patent Publication No. WO 00/40428.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of determining whether a pregnant woman is at elevated risk for carrying a fetus with a chromosomal abnormality, the method comprising
(a) obtaining a biological fluid sample from the woman;
(b) determining the concentration of tissue polypeptide antigen (TPA) in the biological fluid sample; and
(c) using the concentration determined in step (b) for determining whether the pregnant woman is at elevated risk for carrying a fetus with a chromosomal abnormality.

2. The method of claim 1, wherein the determining whether a pregnant woman is at elevated risk for carrying a fetus with a chromosomal abnormality of step (c) is by comparing the concentration determined in step (b) with a control concentration from pregnant women that are not carrying fetuses with a chromosomal abnormality, wherein if the TPA concentration in the biological fluid sample is greater than the control concentration, then the pregnant woman has an elevated risk of carrying a fetus with Down syndrome.

3. The method of claim 1, wherein the determining whether a pregnant woman is at elevated risk for carrying a fetus with a chromosomal abnormality of step (c) is by comparing the concentration determined in step (b) with a control concentration from pregnant women that are not carrying fetuses with a chromosomal abnormality, wherein if the TPA concentration in the biological fluid sample is less than the control concentration, then the pregnant women has an elevated risk of carrying a fetus with a chromosomal abnormality other than Down syndrome.

4. The method of claim 1, wherein the determining whether a pregnant woman is at elevated risk for carrying a fetus with a chromosomal abnormality of step (c) is by comparing the concentration determined in step (b) with a control concentration from pregnant women that are not carrying fetuses with a chromosomal abnormality, wherein if the TPA concentration in the biological fluid sample is not greater or less than the control concentration, then the pregnant women does not have an elevated risk of carrying a fetus with a chromosomal abnormality.

5. The method of claim 1, wherein the fetus has an aneuploidy.

6. The method of claim 1, wherein the fetus has Down syndrome.

7. The method of claim 1, wherein the biological fluid sample is urine or peripheral blood serum.

8. The method of claim 1, wherein the biological fluid sample is peripheral blood serum.

9. The method of claim 1, further comprising subjecting the woman to further testing for carrying a fetus having a chromosomal abnormality if the woman is at elevated risk for carrying a fetus with a chromosomal abnormality.

10. The method of claim 9, wherein the further testing is amniocentesis or chorionic villus sampling.

11. The method of claim 1, further comprising determining the concentration α-fetoprotein (AFP), human chorionic gonadotropin (hCG), unconjugated estriol (uE3), pregnancy-associated plasma protein A (PAPP-A), proform of eosinophilic major basic protein (proMBP), total estrogen, total estriol and/or inhibin A in the biological fluid sample.

12. The method of claim 11, wherein concentrations of AFP, hCG, uE3 and inhibin A are determined.

13. The method of claim 1, wherein the woman is in the first trimester of pregnancy.

14. The method of claim 1, wherein the woman is in the second trimester of pregnancy.

15. The method of claim 1, wherein TPA concentration is estimated by determining the concentration of a component of TPA in the sample.

16. The method of claim 15, wherein the component is keratin 8.

17. The method of claim 15, wherein the component is keratin 18.

18. A kit for determining whether a pregnant woman is at elevated risk for carrying an aneuploid fetus, the kit comprising
a first detectable agent specific for tissue polypeptide antigen (TPA) or a component thereof and
a second detectable agent specific for α-fetoprotein (AFP), human chorionic gonadotropin (hCG), unconjugated estriol (uE3), pregnancy-associated plasma protein A (PAPP-A), proform of eosinophilic major basic protein (proMBP), total estrogen, total estriol or inhibin A.

19. The kit of claim 18, wherein the kit comprises detectable agents specific for AFP, hCG, uEA and inhibin A.

20-25. (canceled)
